(19) United States
(12) Patent Application Publication
(10) Pub. No.: US 2017/0087332 A1
(43) Pub. Date: Mar. 30, 2017

(54) AORTIC CANNULA WITH DE-AIR FUNCTION

(71) Applicant: Corvivo Inc., Richmond (CA)

(72) Inventors: Xiaoming Bian, Dalian (CN); Frank Zheng, Kirkland, WA (US)

(73) Assignee: Corvivo Inc., Richmond (CA)

(21) Appl. No.: 15/375,350

(22) Filed: Dec. 12, 2016

Related U.S. Application Data
(63) Continuation of application No. 13/842,840, filed on Mar. 15, 2013, now abandoned.

(51) Int. Cl. A61M 25/00; A61M 1/36

(52) U.S. Cl. CPC A61M 25/007; A61M 25/0071; A61M 1/3659; A61M 2210/127

(57) ABSTRACT

An aortic cannula comprises a main body portion and a distal end portion. A lumen extends through the main body portion and the distal end portion to allow fluid communication between the main body portion and the distal end portion. The distal end portion has an inner wall and an outer wall. There is a passageway disposed between the inner wall and the outer wall of the distal end portion. The passageway has an inlet and an outlet, and the passageway is fluid communication with a surrounding environment. There may be a stop ring disposed about the distal end portion between the inlet of the passageway and outlet of the passageway.

### RELATED APPLICATIONS

This application claims priority to U.S. patent application Ser. No. 13/842,840, filed Mar. 15, 2013, entitled Aortic Cannula With De-Air Function, which is hereby incorporated herein by reference in its entirety.

### BACKGROUND OF THE INVENTION

Field of the Invention

The present invention relates to a cannula and, in particular, to an aortic cannula that allows for de-airing or venting of the circulatory system during perfusion.

Description of the Related Art

Post-operative venting of the circulatory system following cardiopulmonary bypass surgery is a challenge for cardiac surgeons. Antegrade cardioplegia cannulas have conventionally been used for post-operative venting but it is also known to use aortic cannulas to simultaneously permit bypass blood flow to the heart and venting of the circulatory system. For example, U.S. Pat. No. 6,517,524 discloses a dual lumen aortic cannula that may be used to vent the circulatory system following cardiopulmonary bypass heart surgery. The aortic cannula is constructed and configured to be inserted through a wall of the aorta and simultaneously permits bypass blood flow while venting of the circulatory system using the beating heart as pump.

### SUMMARY OF THE INVENTION

There is accordingly provided an aortic cannula comprising a main body portion, a distal end portion, and a lumen. The lumen extends through the main body portion and the distal end portion to allow fluid communication between the main body portion and the distal end portion. There is a passageway in the distal end portion. The passageway has an inlet and an outlet. The passageway is in fluid communication with the surrounding environment. The distal end portion may have an inner wall and an outer, and the passageway may be disposed between the inner wall and the outer wall. The inlet of the passageway may be an opening which extends through the outer wall of the cannula. The outlet of the passageway may an annular recess in the cannula. There may be a stop ring disposed about the distal end portion between the inlet of the passageway and the outlet of the passageway.

### BRIEF DESCRIPTION OF THE DRAWINGS

The invention will be more readily understood from the following description of the embodiments thereof given, by way of example only, with reference to the accompanying drawings, in which:

FIG. 1 is a perspective view of an improved cannula;

FIG. 2 is a sectional view of the cannula shown in FIG. 1;

FIG. 3 is a partially broken away view of a distal end of the cannula shown in FIG. 1;

FIG. 4 is a fragmentary view of the distal end portion of the cannula shown in FIG. 1 with a top end thereof broken away;

FIG. 5 is a partially broken away view showing the cannula of FIG. 1 cannulating an aorta;

FIG. 6 is a perspective view of another improved cannula; and

FIG. 7 is a sectional view of the cannula shown in FIG. 6.

### DESCRIPTION OF THE PREFERRED EMBODIMENTS

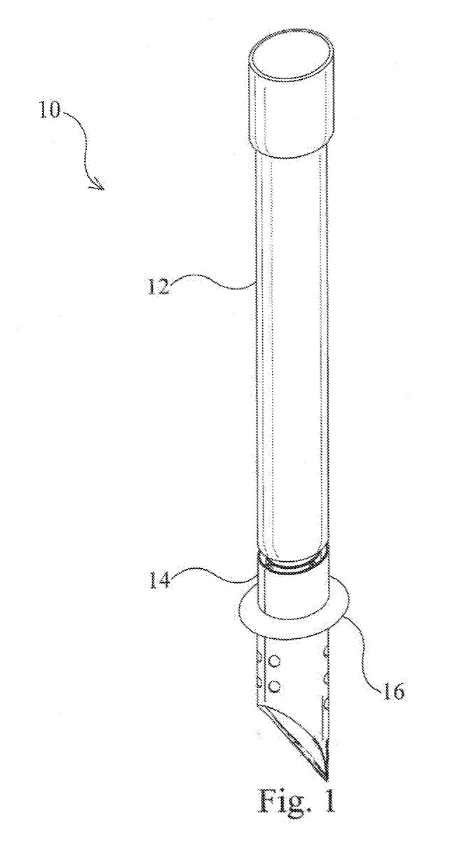

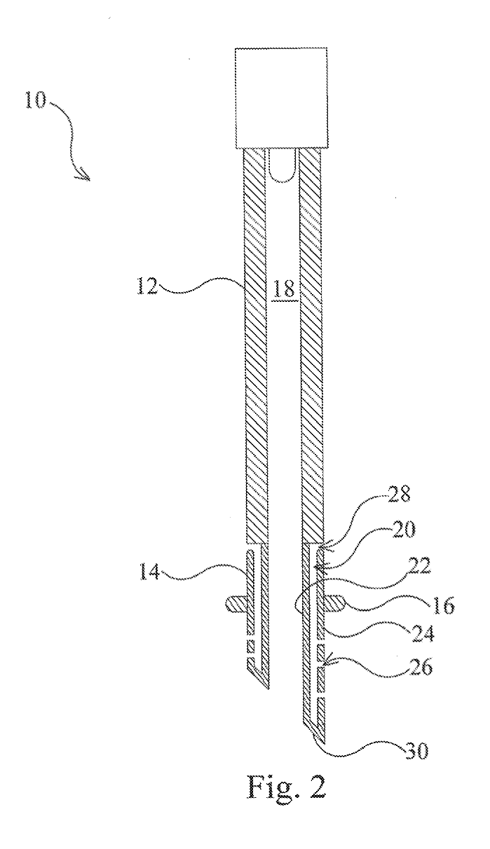

Referring to the drawings and first to FIG. 1, an improved cannula 10 is shown. The cannula 10 generally includes a main body portion 12 and a distal end portion 14. The cannula 10 has a stop ring 16 disposed about the distal end portion 14. The cannula 10 also has a lumen 18 which is best shown in FIG. 2. The lumen 18 extends through both the main body portion 12 and the distal end portion 14 of the cannula 10. The lumen 18 accordingly allows fluid communication between the main body portion 12 and the distal end portion 14 of the cannula 10. The cannula 10, as thus far described, is conventional.

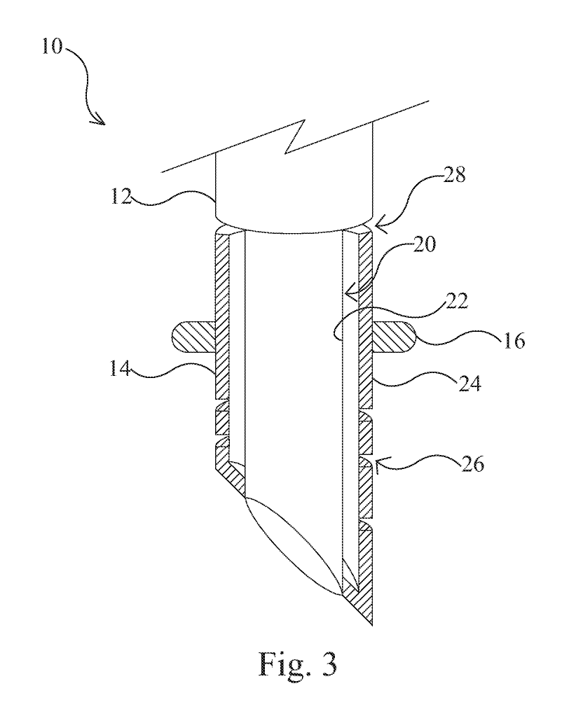

The cannula 10 disclosed herein however is further provided with a plurality of passageways, for example passageway 20 shown in FIG. 2, between an inner wall 22 and an outer wall 24 of the distal end portion 14 thereof. There is a plurality of lateral openings or apertures, for example aperture 26, which extend through the outer wall 24 of the distal end portion 14 to the passageway 20 in the distal end portion 14. The aperture 26 allows fluid communication between the surrounding environment and the passageway 20. There is also an annular opening 28 in the cannula 10 between the main body portion 12 of the cannula and the distal tip portion 14 of the cannula. The annular opening 28 is in fluid communication with the passageway 20 and allows fluid communication between the passageway and the surrounding environment. The above described structure is shown in greater detail in FIG. 3. It may be seen that the opening 28 is a gap between the outer wall 24 of the distal end portion 14 of the cannula 10 and the main portion 12 of the cannula.

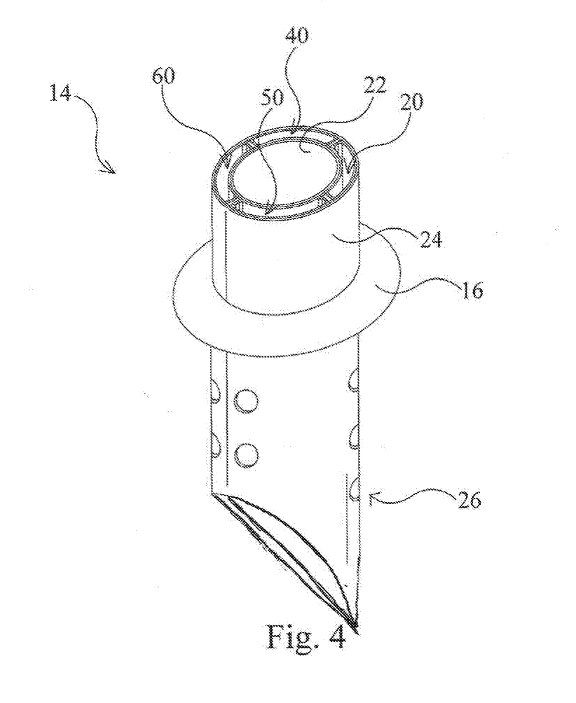

Referring now to FIG. 4, in this example, there are four passageways 20, 40, 50 and 60 in the distal end portion 14 of the cannula 10. As best shown in FIG. 2, for the passageway 20, the passageways extend axially and circumferentially between the inner wall 22 and outer wall 24 from a closed remote end 30 of the distal end portion 14 of the cannula 10 to the annular opening 28 of the cannula. However, in other examples, there may be a single passageway or any number of passageways of varying configurations in the distal end portion of the cannula. The passageways are each in fluid communication with the surrounding environment via respective apertures similar to the aperture 26 for the passageway 20 described above and shown in FIG. 2. Each of the passageways is also in fluid communication with the surrounding environment via the annular opening 28 in the cannula 10 as described above for the passageway 20.

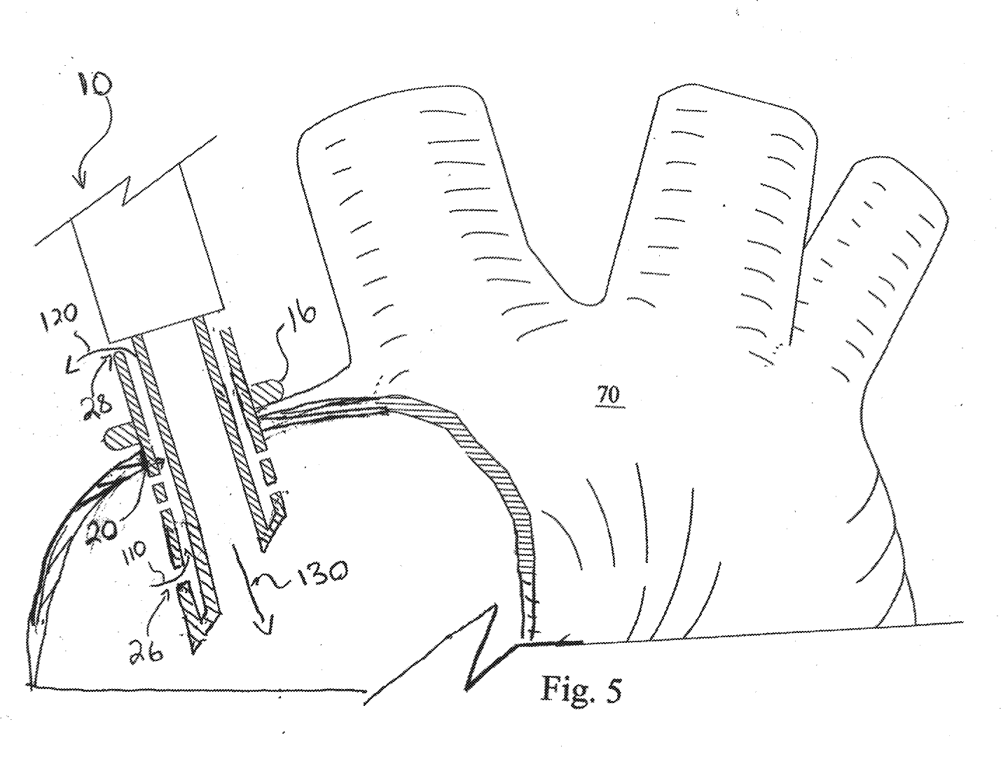

The cannula 10 disclosed herein is useful as an aortic cannula as shown in FIG. 5. The stop ring 16 is disposed between the annular opening 28 in the cannula and the apertures, for example aperture 26, in the distal end portion 14 of the cannula. Fluid from an aorta 70 may accordingly flow through the aperture 26 and similar apertures into the passageways 20, 40, 50 and 60 as generally indicated by arrow 110 when the aorta 70 is cannulated. The fluid then flows along the passageway 20 as generally indicated by arrow 120 and is expelled from the passageway 20 through the annular opening 28. The aperture 26 and similar apertures which extend through the outer wall 24 of the distal end portion 14 of the cannula 10 accordingly function as inlets for air while the annular opening 28 in the cannula 10 functions as an outlet for air to de-air or vent the aorta 70. During de-airing or venting of the aorta 70, perfusion blood may simultaneously flow from the lumen 18 of the cannula 10 into the aorta 70 as generally indicated by arrow 130.

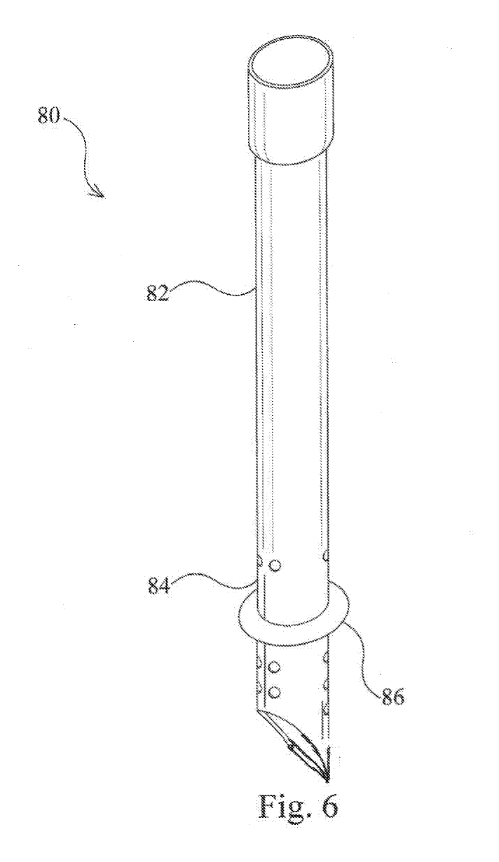

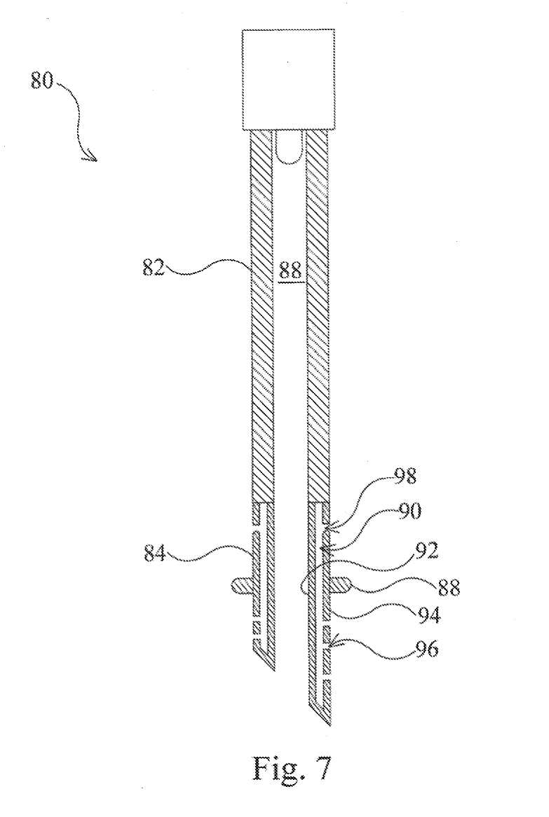

Another improved cannula 80 is shown in FIGS. 6 and 7. The cannula 10 generally includes a main body portion 82 and a distal end portion 84. The cannula 80 has a stop ring 86 disposed about the distal end portion 84. The cannula also has a lumen 88 which is best shown in FIG. 7. The lumen 88 extends through both the main body portion 82 and the distal end portion 84 of the cannula 80. The lumen 88 accordingly allows fluid communication between the main body portion 82 and the distal end portion 84 of the cannula 80.

The cannula 80 is provided with a plurality of passageways, for example passageway 90 shown in FIG. 7, between an inner wall 92 and an outer wall 94 of the distal end portion 84 thereof. There is a plurality of lateral openings or apertures distal of the stop ring 88, for example aperture 96, which extend through the outer wall 94 of the distal end portion 84 to the passageway 90. The aperture 96 allows fluid communication between the surrounding environment and the passageway 90. There is also a plurality of lateral openings or apertures proximal of the stop ring 88, for example aperture 98, which extend through the outer wall 94 of the distal end portion 84 to the passageway 90. The aperture 98 allows fluid communication between the surrounding environment and the passageway 90. The apertures which are distal of the stop ring function as inlets for air while the apertures which are proximal of the stop ring function as outlets for air.

It will be understood by a person skilled in the art that although the cannula disclosed herein is described for use as an aortic cannula that the cannula described herein may be used in other applications.

It will also be understood by a person skilled in the art that many of the details provided above are by way of example only, and are not intended to limit the scope of the invention which is to be determined with reference to the following claims.

What is claimed is:

1. An aortic cannula comprising:
a main body portion;
a distal end portion having an inner wall and an outer wall;
a lumen extending through the main body portion and the distal end portion to allow fluid communication between the main body portion and the distal end portion;
a passageway disposed between the inner wall and the outer wall of the distal end portion, the passageway having an inlet which is an opening which extends through the outer wall of the distal end portion and an outlet which is an opening which extends through the outer wall of the distal end portion, and the passageway being in fluid communication with a surrounding environment; and
a stop ring disposed about the distal end portion between the inlet of the passageway and the outlet of the passageway.

2. An aortic cannula comprising:
a main body portion;
a distal end portion having an inner wall and an outer wall;
a lumen extending through the main body portion and the distal end portion to allow fluid communication between the main body portion and the distal end portion;
a passageway disposed between the inner wall and the outer wall of the distal end portion, the passageway having an inlet which is an opening which extends through the outer wall of the distal end portion and an outlet which is an annular recess in the cannula, and the passageway being in fluid communication with a surrounding environment; and
a stop ring disposed about the distal end portion between the inlet of the passageway and the outlet of the passageway.
